(19) United States
(12) Patent Application Publication
(10) Pub. No.: US 2008/0031836 A1
(43) Pub. Date: Feb. 7, 2008

(54) Crosslinked Nail Varnish Film

(75) Inventor: Philippe Ilekti, Maisons Alfort (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 11/596,812

(22) PCT Filed: May 17, 2005
(86) PCT No.: PCT/FR05/50336

§ 371 Date: Jan. 23, 2007

Related U.S. Application Data
(60) Provisional application No. 60/583,486, filed on Jun. 29, 2004.

(30) Foreign Application Priority Data
May 19, 2004 (FR) 0451005

(51) Int. Cl. A61K 8/84; A61Q 3/02

(52) U.S. Cl. 424/61

(57) ABSTRACT

The invention relates to a flexible article which is used to make up and/or care for nails and/or false nails, comprising: at least one adhesive layer which is used to fix the article to the nail, and at least one cross-linked film.

The present invention relates to a flexible article intended to be applied to the nails for making them up and/or caring for them.

Conventionally, the making up of nails or false nails is performed using liquid makeup compositions, also commonly known as nail varnishes. This nail varnish is generally applied in the form of layers superposed at the surface of the nail to be made up, an intermediate drying step being observed between each applied layer of varnish. In point of fact, this makeup method proves not to be entirely satisfactory.

Firstly, its application requires a certain amount of time.

Moreover, this type of makeup needs to be reapplied at short intervals on account of its insufficient staying power. Specifically, very quickly, generally after three to five days, the applied varnish flakes off and its gloss reduces. It is then necessary to perform a makeup removal step and to repeat a new makeup operation.

Finally, standard nail varnish formulations generally involve the use of volatile solvents, which give rise, during the application, to an unpleasant odor.

Several alternatives have already been proposed in an attempt to, at least partly, overcome the abovementioned drawbacks. Thus, nail makeup products have been proposed in the form of a kit of two liquid nail varnish compositions. However, the improvement in staying power is acquired in this case at the expense of the application conditions, which double the number of layers to be applied.

Another alternative consisted in developing nail varnish compositions based on a dispersion of polymers in aqueous phase, which are thus satisfactory in olfactory terms. Unfortunately, the corresponding varnishes prove to have insufficient staying power over time.

The present invention is specifically directed towards proposing a method for making up and/or caring for the nails or false nails, which is precisely, as opposed to standard liquid formulations of nail varnish type, easy to apply and has significantly improved staying power over time and a markedly reduced content of organic solvent(s).

More precisely, according to a first of its aspects, the present invention relates to a flexible article for making up and/or caring for the nails and/or false nails, comprising:

at least one adhesive layer for fixing the article to the nail, and

at least one crosslinked film, which is especially colored or transparent.

In one variant, the article may comprise a superposition of at least two or even more different crosslinked films.

In particular, one of the films, generally the one in contact with the adhesive layer, may be colored and the other transparent.

According to another variant of the invention, the article may also comprise, between the adhesive layer and the crosslinked film, at least one film of colored varnish. Such a film may be derived from the evaporation of the organic or aqueous solvent phase of a solution or dispersion, which is generally colored, of at least one film-forming polymer. This film-forming polymer may be chosen especially from nitrocellulose and cellulose esters.

According to this variant of the invention, the crosslinked film is preferably transparent.

As used herein, the term “transparent” means that the crosslinked layering has a Hazebyk index of less than 5 as measured using a Kykhazegloss glossmeter.

According to a second aspect, the present invention relates to a process for preparing a flexible article for making up and/or caring for the nails, comprising at least the steps consisting in superposing on a removable support:

a) at least one layer of a composition based on at least one adhesive material, and

b) at least one layer of a crosslinkable composition, the crosslinking of said composition being performed consecutively to its deposition so as to obtain a crosslinked film.

According to a first variant of the invention, the process comprises at least the steps consisting in:

a) depositing on a removable support at least one layer of a composition containing at least one adhesive material,

b) depositing on said adhesive layer at least one layer of a crosslinkable composition,

c) crosslinking said composition so as to obtain a crosslinked film, and

d) if necessary, at least partially drying said article.

According to a second variant of the invention, the process comprises at least the steps consisting in:

a) depositing on a removable support at least one layer of a crosslinkable composition,

b) crosslinking said composition so as to obtain a crosslinked film,

c) if necessary, at least partially drying said film,

d) depositing on said film obtained in c) at least one layer of a composition containing at least one adhesive material,

e) if necessary, at least partially drying said article,

f) covering the adhesive layer obtained in e) with a removable support and, where appropriate,

g) recovering said article by peeling the crosslinked film from the support in a).

According to another variant of the invention, said process also comprises at least one step that consists in forming between the adhesive layer and the crosslinked film a film of colored varnish.

According to a third aspect, the present invention relates to a product for making up and/or caring for the nails and/or false nails, comprising, in a substantially airtight packaging, at least one article in accordance with the invention, the packaging being such that the article is conserved therein in a partially dry form.

For the purposes of the present invention, the term “partially dry” is intended to denote the fact that the article obtained after forming the crosslinked film is not totally free of the residual solvent. In particular, it has a solids content of less than 80%, in particular less than 75% and more particularly less than 70% by weight relative to its total weight.

According to one particular embodiment, this packaging comprises a reservoir, for instance a flexible or inflexible pocket, capable of containing a product in a leaktight manner to preserve said article from total and premature drying out before its use. More specifically, the packaging is leaktight to air and/or to solvents.

According to a fourth aspect, the present invention relates to a process for preparing a product as defined above, comprising the steps consisting in superposing on a removable support:

at least one layer of a composition based on at least one adhesive material,

at least one layer of a crosslinkable composition, the crosslinking being performed consecutively to the deposition of said composition,

if necessary, partial drying of said article thus obtained, and

packaging of said article in a partially dry form in substantially airtight packaging.

According to this embodiment, the article does not acquire a totally dry appearance, and thus its definitive form, until after application to the nail, by simple exposure to the ambient air.

According to a fifth aspect, the present invention relates to a process for making up and/or caring for the nails, comprising the fact that the adhesive face of an article according to the invention is applied to a nail.

In general, the article according to the present invention is in the form of a film.

For the purposes of the present invention, the term “flexible” qualifies a sufficient flexibility of this film, i.e. flexibility that is suitable for mechanical deformations of stretching type to adjust it to the surface of a nail. This deformability is especially characterized by the ultimate strain εᵣ discussed hereinbelow.

The article according to the invention differs especially in this respect from an article of false nail type, which is characterized by a rigidity that is incompatible with such a mechanical deformation.

Another difference between the article in accordance with the invention and a false nail lies in the sensitivity of this article with respect to polar organic solvents such as acetone and short esters and/or alcohols. Specifically, the crosslinked film on the outer face of the article according to the invention, i.e. the face not adhering to the nail, has a tendency to increase in volume and thus in weight when it is placed in contact with one of these solvents. A false nail is totally devoid of such sensitivity. This swellability manifested by the article according to the invention is precisely advantageous for its removal when it is applied to the surface of a nail or a false nail. The reason for this is that the article according to the invention may be readily removed by simple makeup removal with a standard dissolver, as opposed to a false nail that becomes detached. Thus, the article according to the invention can advantageously be removed with organic solvents and especially with alkyl acetates and mixtures thereof.

The article in accordance with the invention also has significant staying power over time, especially on a scale of at least one week. It thus proves to be resistant to water, rubbing and impacts, and does not show any significant wear or chipping in this period.

The article according to the invention may be used either for makeup purposes, in which case it generally comprises at least one colored crosslinked film, or for protective purposes with respect to a film of varnish. In this alternative, it generally comprises a transparent crosslinked film.

### I. ARTICLE ACCORDING TO THE INVENTION

The article according to the invention may be characterized by a high dry extract. Specifically, the amount of solids is greater than 80%, in particular greater than 85% and in particular greater than 90% by weight relative to the total weight of the article. In other words, the amount of volatile solvent is less than 20%, in particular less than 15% and more particularly less than 10% by weight relative to the total weight of the article.

However, according to another preferred embodiment, the article according to the invention may advantageously be in a partially dry form. In this particular case, the article is packaged in a reservoir, for instance a flexible or inflexible pocket, which is sufficiently leaktight for it to preserve this partially dry appearance. It is not until the time of its use, and consequently of placing it in contact with air, that the article dries out totally to acquire the solids content described previously.

In a product according to the invention, the article according to the invention advantageously has a solids content of less than 80%, especially less than 75% and more particularly less than 70% by weight relative to its total weight. Said article may moreover have a solids content of greater than 60% and especially greater than 65% by weight relative to its total weight. When it is extracted from the packaging of a product in accordance with the invention and exposed to the ambient air, such an article acquires a dry state as defined above after 24 hours.

Preferably, the amount of solids, commonly referred to as the “dry extract”, of the articles according to the invention is measured by heating the sample with infrared rays with a wavelength of from 2 μm to 3.5 μm. The substances contained in said films that have a high vapor pressure evaporate under the effect of this irradiation. Measurement of the weight loss of the sample makes it possible to determine the “dry extract” of the article. These measurements are performed using an LP16 commercial infrared desiccator from Mettler. This technique is fully described in the machine documentation supplied by Mettler.

The measuring protocol is as follows.

About 10 g of sample of an article are deposited on a metal crucible. After placing it in the desiccator, this crucible is subjected to a nominal temperature of 120° C. for one hour. The wet mass of the sample, corresponding to the initial mass, and the dry mass of the sample, corresponding to the mass after exposure to radiation, are measured using a precision balance.

The solids content is calculated in the following manner:
Dry extract=100×(dry mass/wet mass).

Water Uptake

The article according to the invention may be characterized in dry form by an uptake of water brought to 25° C. of less than or equal to 20%, especially less than or equal to 16% and in particular less than 10%.

According to the present patent application, the term “water uptake” means the percentage of water absorbed by the article after 60 minutes of immersion in water at 25° C. (room temperature). The water uptake is measured for pieces of about 1 cm² cut from the dry article. They are weighed (mass measurement M1) and then immersed in the water for 60 minutes; after immersion, the piece of film is wiped to remove the excess surface water and then weighed (mass measurement M2). The difference M2−M1 corresponds to the amount of water absorbed by the film.

The water uptake is equal to [(M2−M1)/M1]×100 and is expressed as a weight percentage relative to the weight of the film.

Storage Modulus E′

Moreover, the article according to the invention is advantageously a film with a storage modulus E′ of greater than or equal to 1 MPa, especially ranging from 1 MPa to 5000 MPa, in particular greater than or equal to 5 MPa, especially ranging from 5 to 1000 MPa, and more particularly greater than or equal to 10 MPa, for example ranging from 10 to 500 MPa, at a temperature of 30° C. and a frequency of 0.1 Hz.

The storage modulus is measured by DMTA (Dynamic and Mechanical Temperature Analysis).

Viscoelasticimetry tests are performed with a DMTA machine from Polymer TA Instruments (model DMA2980) on a sample of article. The specimens are cut out (for example using a sample punch). These specimens are typically about 150 μm thick, from 5 to 10 mm wide and have a useful length of about 10 to 15 mm.

The measurements are taken at a constant temperature of 30° C.

The sample is subjected to tension and to small bending (for example, a sinusoidal displacement of ±8 μm is imposed thereon) during a frequency scan, the frequency ranging from 0.1 to 20 Hz. The test is thus performed in the linear region, at low levels of bending.

These measurements make it possible to determine the complex modulus E*=E′+iE″ of the test film of composition, E′ being the storage modulus and E″ the loss modulus.

Ultimate Strain and/or Energy at Break

Advantageously, the articles according to the invention have an ultimate strain εᵣ of greater than or equal to 5%, especially ranging from 5% to 500%, preferably greater than or equal to 15% and especially ranging from 15% to 400%, and/or an energy at break per unit volume Wᵣ of greater than or equal to 0.2 J/cm³, especially ranging from 0.2 to 100 J/cm³, preferably greater than 1 J/cm³ and especially ranging from 1 to 50 J/cm³.

The ultimate strain and the energy at break per unit volume are determined by tensile tests performed on a crosslinked film about 200 μm thick.

To perform these tests, the article is cut into dumbbell-shaped specimens with a useful length of 33±1 mm and a useful width of 6 mm. The cross section (S) of the specimen is then defined as being: S=width×thickness (cm²); this cross section will be used for calculating the stress.

The tests are performed, for example, on a tensile testing machine sold under the name Lloyd® LR5K. The measurements are performed at room temperature (20° C.).

The specimens are pulled at a traveling speed of 33 mm/minute, corresponding to a rate of 100% elongation per minute.

A traveling speed is thus imposed and the elongation ΔL of the specimen and the force F required to impose this elongation are simultaneously measured. From these data ΔL and F, the stress a and strain ε parameters are determined.

A curve is thus obtained of stress σ=(F/S) as a function of the strain ε=(ΔL/Lₒ)×100, the test being performed up to the breaking point of the specimen, Lₒ being the initial length of the specimen.

The ultimate strain εᵣ is the maximum deformation of the sample before the breaking point (in %).

The energy at break per unit volume Wᵣ in J/cm³ is defined as the area under this stress/strain curve such that:

$$Wr = {\int }_{0}^{{ɛ}_{r}} σ \cdot ɛ \cdot ⅆ ɛ$$

Crosslinked Film

As stated previously, the film according to the invention is derived from crosslinking.

For the purposes of the present invention, a film termed as being crosslinked may be totally or partially crosslinked.

In the case of partial crosslinking, this crosslinking is, of course, sufficient to form the expected film.

This crosslinking may thus be performed thermally, photochemically and/or chemically, in the presence or absence of a catalyst. Performing this crosslinking is within the competence of a person skilled in the art.

Needless to say, the compounds placed in contact for the crosslinking are chosen, especially according to the nature of the reactive functions they respectively bear, so as to be capable of interacting under the crosslinking reaction conditions under consideration.

According to a first variant, the crosslinking reaction is likened to a polyaddition or polycondensation reaction performed in the presence or absence of catalyst.

In this particular case, the crosslinkable composition contains at least one reactive system formed by:

at least one first compound (A) comprising at least two functions X, and

at least one second compound (B) comprising at least two functions Y, that are reactive with the functions X.

Advantageously, the reactive system has a mean functionality (total number of functions X and Y/total number of molecules of compounds (A) and (B)) of greater than 2 so as to afford a three-dimensional network.

More particularly, to obtain a satisfactory crosslinking effect, the mean functionality of the reactive system may be at least equal to 2.2 and more particularly range from 2.5 to 100.

The compounds (A) and (B) may be of organic origin and especially of oligomer, polymer and/or copolymer type, or of inorganic origin such as, for example, a mineral particle, in which case they bear at the surface the two required functions X or Y.

The functions X and Y that are reactive with each other are chosen from “reactive” functions and functions comprising at least one labile hydrogen.

More specifically, the reactive functions are chosen from isocyanate, epoxide and ethylenic double bond functions and the functions containing labile hydrogen(s) are of the carboxylic, alcohol, especially phenol, primary or secondary amine, amide, amino alcohol and/or thiol type.

More particularly, the compounds (A) and (B) placed in contact respectively bear at least two “reactive” functions, especially of epoxide and/or isocyanate type, and at least two functions containing labile hydrogen(s) especially of amine or amino alcohol type, and may be chosen especially from the compounds mentioned above.

For example, X may be an epoxide and/or isocyanate function and Y may be chosen from a carboxylic acid function and/or an anhydride function and/or an amine function and/or a thiol function and/or a hydroxyl function, in particular a phenol function.

Compounds Containing Isocyanate Functions:

Compounds comprising at least two free isocyanate functions are known in the art. They may be polyisocyanates, including diisocyanates or triisocyanates, which may have a molecular mass of less than 500 000 and especially less than 10 000. These polyisocyanates are generally obtained by polyaddition, polycondensation and/or grafting, bearing at least two isocyanate functions, either at the chain ends or on side groups.

The polyisocyanates may be linear, branched, aliphatic, cycloaliphatic or aromatic.

Among the compounds of this type that may be used, mention may be made of:

a) Diisocyanates containing from 4 to 50 and especially from 4 to 30 carbon atoms, such as 1,4-tetramethylene diisocyanate, 1,6-hexamethylene diisocyanate, 2,6- and 2,4-toluene diisocyanate, 5 diphenylmethane diisocyanate and isophorone diisocyanate,

b) the triisocyanates of formulae: 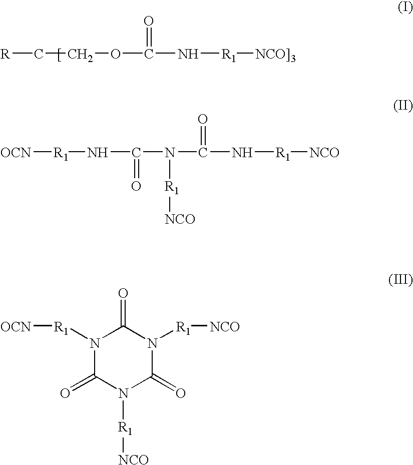
in which R is an alkyl radical containing from 1 to 30 carbon atoms, each R₁ independently representing a linear, branched or cyclic divalent hydrocarbon-based radical containing from 2 to 30 carbon atoms,

c) polycondensates containing isocyanate end or side groups, such as polyurethanes and/or polyureas (including block copolymers comprising at least one polyurethane and/or polyurea block and at least one polyether, polyester, polysiloxane, alkyd or polyacrylate block), and also polyesters, polyamides, polyepoxy, polyethers and perfluoropolyethers, and

d) polymers resulting from the copolymerization of vinyl, allylic and/or (meth)acrylic monomers and of ethylenically unsaturated comonomers comprising a free isocyanate function, for instance 2-isocyanatoethyl methacrylate.

Polyisocyanates that may be used in particular include Desmodur® N from the company Bayer or Tolonate® HDB-LV from the company Rhodia.

Compounds Containing Epoxide Functions:

Compounds comprising at least two epoxide functions are also known in the prior art. They may be of any chemical nature. They may be diepoxides or poly-epoxides of low mass (less than or equal to 5000), or alternatively oligomers or polymers of any chemical nature, obtained by polyaddition, polycondensation and/or grafting, bearing at least two free epoxide functions, either at the chain ends or as side groups.

Examples of such compounds that may be mentioned include:

a) bisphenol A diglycidyl ether resulting from the condensation between bisphenol A and epichlorohydrin, of structure

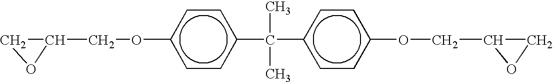

b) diepoxy resins resulting especially from the higher condensation between bisphenol A diglycidyl ether and epichlorohydrin,

c) epoxy ester resins containing α, ω-diepoxy end groups, especially resulting from the condensation of a dicarboxylic acid especially containing from 2 to 60 carbon atoms with a stoichiometric excess of compounds a) or b),

d) epoxy ether resins containing α,ω-diepoxy end groups, especially resulting from the condensation of a diol especially containing from 2 to 60 carbon atoms with a stoichiometric excess of compounds a) or b),

e) natural or synthetic oils bearing at least two epoxide groups, for instance epoxidized soybean oil, epoxidized linseed oil and vernonia oil, which are described especially in patent application EP-A-645 134,

f) oligomers or polymers resulting from the copolymerization of unsaturated or vinyl, allylic and/or (meth)acrylic monomers, and of ethylenically unsaturated comonomers comprising a free epoxide function (for instance glycidyl methacrylate), and

g) other polycondensates containing epoxy end and/or side groups, such as polyesters, polyester-amides, polyamides, alkyds, polyurethanes and/or polyureas, polyethers and perfluoropolyethers or silicones.

Polymers containing epoxy functions are sold under the names Cyracure® UVR-6110, Cyracure® UVR-6105, Cyracure® ERL-4221E, Cyracure® ERL-4206, Cyracure® UVR 6128, Cyracure® UVR 6216 by the company Union Carbide, DER® 439 by the company Dow Chemical, Epikates® 828,1001,1004,1007 from the company Shell, Araldite® ECN1299 from the company Ciba-Geigy and Epoxynovolacs® from the company Dow Chemical.

Compounds Containing Ethylenic Double Bonds:

The compounds bearing ethylenic double bonds may be of any chemical nature. They may be chosen especially from:

a) ethylenically unsaturated polyesters.

This is a group of polymers of polyester type containing one or more ethylenic double bonds randomly distributed in the main chain of the polymer.

These unsaturated polyesters are obtained by polycondensation of a mixture:

of linear or branched aliphatic or cycloaliphatic dicarboxylic acids especially containing from 3 to 50 carbon atoms and preferably from 3 to 20 carbon atoms, such as adipic acid or sebacic acid, aromatic dicarboxylic acids especially containing from 8 to 50 carbon atoms and preferably from 8 to 20 carbon atoms, such as phthalic acids, especially terephthalic acid, and/or dicarboxylic acids derived from ethylenically unsaturated fatty acid dimers such as the oleic or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexanedimethanol, of aromatic diols containing from 6 to 50 carbon atoms and preferably from 6 to 20 carbon atoms, such as bisphenol A, bisphenol B, and/or of diol dimers derived from the reduction of the fatty acid dimers as defined above, and

of one or more dicarboxylic acids or anhydrides thereof comprising at least one polymerizable ethylenic double bond and containing from 3 to 50 carbon atoms and preferably from 3 to 20 carbon atoms, such as maleic acid, fumaric acid or itaconic acid;

b) polyesters containing (meth)acrylate side and/or end groups:

This is a group of polymers of polyester type obtained by polycondensation of a mixture:

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexanedimethanol, of aromatic diols containing from 6 to 50 carbon atoms and preferably from 6 to 20 carbon atoms, such as bisphenol A and bisphenol B, and

of at least one monoester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate and glyceryl methacrylate.

These polyesters differ from those described above in point a) in that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50000: molar mass 1500, on average 6 acrylate functions per molecule);

c) polyurethanes and/or polyureas containing (meth)acrylate groups obtained especially by polycondensation:

of aliphatic, cycloaliphatic and/or aromatic diisocyanates, triisocyanates and/or polyisocyanates especially containing from 4 to 50 and preferably from 4 to 30 carbon atoms, such as hexamethylene diisocyanate, isophorone diisocyanate, toluene diisocyanate, diphenylmethane diisocyanate or the isocyanurates of formula: 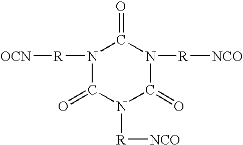
resulting from the trimerization of 3 diisocyanate molecules OCN—R—CNO, in which R is a linear, branched or cyclic hydrocarbon-based radical containing from 2 to 30 carbon atoms;

of polyols, especially diols, free of polymerizable ethylenic unsaturations, such as 1,4-butanediol, ethylene glycol or trimethylolpropane, and/or of polyamines, especially aliphatic, cycloaliphatic and/or aromatic diamines especially containing from 3 to 50 carbon atoms, such as ethylenediamine or hexamethylenediamine, and

of at least one monoester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate and glyceryl methacrylate.

Such polyurethane/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molar mass 1500, 2 acrylate functions per molecule, Ebecryl® 230: molar mass 5000, 2 acrylate functions per molecule, Ebecryl® 270: molar mass 1500, 2 acrylate functions per molecule, Ebecryl® 8402: molar mass 1000, 2 acrylate functions per molecule, Ebecryl® 8804: molar mass 1300, 2 acrylate functions per molecule, Ebecryl® 220: molar mass 1000, 6 acrylate functions per molecule, Ebecryl® 2220: molar mass 1200, 6 acrylate functions per molecule, Ebecryl® 1290: molar mass 1000, 6 acrylate functions per molecule, Ebecryl® 800: molar mass 800, 6 acrylate functions per molecule).

Mention may also be made of the water-soluble aliphatic polyurethane diacrylates sold under the names Ebecryl® 2000, Ebecryl® 2001 and Ebecryl® 2002, and the polyurethane diacrylates in aqueous dispersion sold under the trade names IRR® 390, IRR® 400, IRR® 422 IRR® 424 by the company UCB;

d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley;

e) epoxy acrylates obtained by reaction between:

at least one diepoxide chosen, for example, from:
1) bisphenol A diglycidyl ether,
2) a diepoxy resin resulting from the reaction between bisphenol A diglycidyl ether and epichlorohydrin,
3) an epoxy ester resin containing α,ω-diepoxy end groups resulting from the condensation of a dicarboxylic acid containing from 3 to 50 carbon atoms with a stoichiometric excess of 1) and/or 2), and
4) an epoxy ether resin containing α,ω-diepoxy end groups resulting from the condensation of a diol containing from 3 to 50 carbon atoms with a stoichiometric excess of 1) and/or 2),
5) natural or synthetic oils bearing at least two epoxide groups, such as epoxidized soybean oil, epoxidized linseed oil and epoxidized vernonia oil,
6) a phenol-formaldehyde polycondensate (Novolac® resin) whose end groups and/or side groups have been epoxidized, and

one or more carboxylic acids or polycarboxylic acids comprising at least one ethylenic double bond alpha to the carboxylic group, for instance (meth)acrylic acid, crotonic acid or monoesters of (meth)acrylic acid and of a diol or polyol containing from 2 to 20 carbon atoms and preferably from 2 to 6 carbon atoms, such as 2-hydroxyethyl (meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600 and Ebecryl® 609, Ebecryl® 150, Ebecryl® 860 and Ebecryl® 3702 by the company UCB and under the names Photomer® 3005 and Photomer® 3082 by the company Henkel;

f) poly(C₁₋₅₀ alkyl) (meth)acrylates comprising at least two ethylenic double bond functions borne by the hydrocarbon-based side and/or end chains.

Such copolymers are sold, for example, under the names IRR® 375, OTA® 480 and Ebecryl® 2047 by the company UCB;

g) polyorganosiloxanes containing (meth)acrylate or (meth)acrylamide groups, obtained especially, respectively:

by esterification, for example of (meth)acrylic acid and of polyorganosiloxanes, especially of polydimethylsiloxanes (PDMS), bearing hydroxyl end and/or side groups,

by amidation, for example of (meth)acrylic acid and of polyorganosiloxanes bearing primary or secondary amine side and/or end groups.

Among the hydroxylated PDMSs that may be mentioned are PDMSs comprising at least two C₁₋₆ hydroxyalkyl groups and dimethicone copolyols with hydroxyl side or end groups.

Esterifiable α,ω-dihydroxylated polydimethylsiloxanes are sold under the names Tegomer® H—Si 2111 and Tegomer® H—Si 2311 by the company Goldschmidt. α,ω-Diacrylate polydimethylsiloxanes are available from the company Shin-Etsu under the references X-22-164 B and X-22-164C.

Amino PDMSs that may be mentioned in particular are PDMSs comprising at least 2 C₁₋₁₀ aminoalkyl groups, for example the amino silicone sold under the name Q2-8220 by the company Dow Corning.

Advantageously, the silicone polymers of this group are used as a mixture with one or more polymers of the other groups a) to f) described above, especially to modify the hydrophobic nature of the final composition;

h) perfluoropolyethers containing acrylate groups obtained especially by esterification, for example with (meth)acrylic acid, of perfluoropolyethers bearing hydroxyl side and/or end groups.

Such perfluoropolyether α,ω-diols are described especially in EP-A-1 057 849 and are sold by the company Ausimont under the name Fomblin® Z Diol;

i) dendrimers and hyperbranched polymers bearing (meth)acrylate or (meth)acrylamide end groups, obtained especially, respectively, by esterification or amidation of hyperbranched dendrimers and polymers containing hydroxyl or amino end functions, with (meth)acrylic acid.

Dendrimers (from the Greek dendron=tree) are “arborescent” polymeric molecules, i.e. highly branched molecules, invented by D. A. Tomalia and his team in the early 1990s (Donald A. Tomalia et al., Angewandte Chemie, Int. Engl. Ed., vol. 29, No. 2, pages 138-175). They are structures constructed around a central unit that is generally polyvalent. Around this central unit are connected, in a fully defined structure, branched chain-extending units, thus giving rise to monodispersed symmetrical macromolecules having a well defined chemical and stereochemical structure. Dendrimers of the polyamidoamine type are sold, for example, under the name Starbust® by the company Dendritech.

Hyperbranched polymers are polycondensates, generally of polyester, polyamide or polyethyleneamine type, obtained from multifunctional monomers, which have an arborescent structure similar to that of dendrimers but much less regular than the latter (see for example WO-A-93/17060 and WO 96/12754).

The company Perstorp sells hyperbranched polyesters under the name Boltorn®. Hyperbranched polyethyleneamines are available under the name Comburst® from the company Dendritech. Hyperbranched poly(esteramides) with hydroxyl end groups are sold by the company DSM under the name Hybrane®.

These dendrimers and hyperbranched polymers esterified or amidated with acrylic and/or methacrylic acid are distinguished from the polymers described in points a) to h) above by the very large number of ethylenic double bonds present.

This high functionality, usually greater than 5, makes them particularly useful by allowing them to act as a “crosslinking node”, i.e. a site of multiple crosslinking.

Compounds Bearing at Least Two Functions Containing Labile Hydrogen(s)

Compounds bearing at least two functions containing labile hydrogen used in the present invention are also known. These may be organic compounds of low molecular mass or synthetic oligomers or polymers, obtained by polyaddition, polycondensation and/or grafting, or chemically modified natural polymers.

According to the present invention, the functions containing labile hydrogen are preferably chosen from primary amine (—NH₂), secondary amine (>NH), hydroxyl (—OH), carboxylic acid (—COOH) and thiol (—SH) functions.

When the function containing labile hydrogen is a hydroxyl function, families of compounds that may be mentioned include diols and polyols, preferably chosen from:

1) aliphatic diols containing a hydroxyl group, such as 1,2-propanediol, 1,3-propanediol, 1,2-butanediol, 1,3-butanediol, 1,2-pentanediol, 1,4-pentanediol, etc.,

2) polyols such as polyalkylene ether polyols whose structure is described by the formula:
H—(O(CH₂—CHR)ₙ)ₘOH
with:

R being a hydrogen atom or an alkyl group containing from 1 to 5 carbons,

n being an integer from 1 to 6, and

m being an integer from 1 to 100.

Mention may be made in particular of poly(oxytetraethylene) glycol, poly(oxy-1,2-propylene) glycol, poly(oxy-1,2-butylene) glycol, etc.

When the function containing labile hydrogen is an amine function (NH₂), it may be a diamine, a polyamine, an amino alcohol, or an oligomer or polymer containing amine groups, especially chosen from:

1) aliphatic, cycloaliphatic or aromatic diamines especially containing from 2 to 60 carbon atoms, such as ethylenediamine, 1,2-diaminopropane, 1,3-diaminopropane, 1,4-diaminobutane, 1,2-diamino-2-methylpropane, 1,6-diaminohexane, 1,10-diaminodecane, isophoronediamine, adamantanediamine, 2,6-diamino-pyridine, or the diamines obtained by modification of the end groups of fatty acid dimers,

2) multifunctional amines containing more than two amine groups, such as melanine, 2,4,6-triamino-pyrimidine, 3,3′-diaminobenzidine or 2,4,5,6-tetra-aminopyrimidine,

3) oligomers bearing at least two amine groups, such as the Jeffamine® polyalkylene oxide diamines from Texaco (polyetherdiamines),

4) dendrimers or hyperbranched polymers whose chain end groups are primary amines, in particular polyamidoamines such as those sold under the name Starbust® by the company Dendritech. Hyperbranched polymers are polycondensates generally of polyethyleneamine type obtained from multifunctional monomers, which have an arborescent structure similar to that of dendrimers but much less regular than the latter.

When the compound is an oligomer or polymer (hompolymer or copolymer) bearing functions containing labile hydrogen, these functions may be located at the chain ends and/or laterally on the chains. Families of compounds that may be mentioned include block or non-block acrylic copolymers resulting from the polymerization of monomers comprising unsaturated ethylenic functions such as (meth)acrylic acid with a (meth)acrylic acid alkyl ester monomer (methyl methacrylate, ethyl methacrylate, etc.), vinyl monomers such as styrene, α-methyl styrene, vinyltoluene, polyurethanes, polyesters, polycondensates of any nature bearing hydroxyl groups, and in particular polyamides obtained by condensation of an excess of diamine or of diacid or of diol.

Particular examples of compounds bearing functions containing labile hydrogens are: C₁₋₄ alkylene glycols, glycerol, trimethylolpropane, pentaerythritol, poly(C₁₋₄ alkylene) glycols such as polyethylene glycol or polypropylene glycol or copolymers thereof, the product of condensation of propylene glycol and of trimethylolpropane, castor oil, phytanetriol, sugars and carbohydrates such as sucrose or cellulose, ethylenediamine, 1,3-diaminopropane, lysine, 2-amino-2-methyl-1-propanol, poly(alkyleneoxy)diamines such as the Jeffamine® products sold by the company Texaco, nitrocellulose, cellulose esters, especially those with a degree of substitution of less than 3, such as cellulose acetobutyrate and cellulose acetopropionate, cellulose ethers such as hydroxyethylcellulose, carboxymethylcellulose, hydroxypropylcellulose or ethylcellulose, polyester resins, silicones, perfluoropolyethers, alkyds and polyketones containing hydroxylated end groups, poly(vinyl alcohol) and copolymers based on vinyl alcohol, allylic alcohol copolymers, copolymers based on C₂₋₁₀ hydroxyalkyl (meth)acrylate, for instance 2-hydroxyethyl or 2-hydroxypropyl (meth)acrylate, sold especially under the name Joncryl® SCX 910 by the company Johnson Polymer or under the name Crodoplast® AC 5725 by the company Croda, copolymers based on vinylamine or allylamine, silicones and perfluoroethers containing primary or secondary amine end groups, dendrimers or hyperbranched polymers containing hydroxyl or primary amine end groups such as hyperbranched polyesters containing hydroxyl end groups sold by the company Perstorp under the names Boltorn® H40 TMP Core and HBP Polyol® 3G (described in international patent applications WO 93/17060 and WO 96/12754), or dendrimers of polyamidoamine type containing primary amine end groups described in the article by Tomalia, Angewandte Chemie, Int. Engl. Ed., vol. 29, No. 2, pages 138-175.

Compounds bearing at least two functions of different nature, for instance resins, are also suitable for the invention. These may especially be:

alkyd resins, and in particular oligomers derived from the condensation of compounds bearing hydroxyl functions (alcohol, diol, etc.) with compounds bearing carboxylic acid functions.

Thus, alkyd resins comprise functions containing labile hydrogens such as hydroxyl functions and/or amide functions and/or carboxylic acid functions and/or reactive functions such as unsaturated ethylenic functions (derived from an alcohol or a fatty acid containing an alkyl chain comprising an unsaturated ethylenic function). These resins may be used as compound A or B to be crosslinked in the presence of a compound comprising a reactive function such as an isocyanate or an epoxide function, or co-crosslinked by UV with another compound comprising unsaturated ethylenic functions;

aminoplast resins resulting from the condensation of a compound containing an aldehyde function with a compound containing an amine or amide function.

Examples that may be mentioned include reactions of condensation of formaldehyde, acetaldehyde or benzaldehyde with a urea or a melamine. The preferred aminoplast resins are derived from the condensation of an alcohol function and formaldehyde with a urea or melamine function.

According to a second variant of the invention, the crosslinking is performed photochemically and involves at least two compounds (A) and (B) bearing functions of unsaturated double bond type, in the presence of a photoinitiator.

According to this variant, the compounds (A) and (B) are chosen so as to form a reactive system in which the mean valency of the system is greater than 2.

The term “valency of a compound” means the number of covalent bonds it can establish with the compounds associated therewith. The mean valency is defined as being equal to the ratio of the sum of the valencies of all the compounds (A) and (B) divided by the total number of compounds (A) and (B)

$${V}_{m} = \frac{\sum nivi}{\sum ni}$$

According to this variant of the invention, the compounds (A) or (B) may be a compound comprising a function of unsaturated double bond type and especially as defined previously, and/or an ethylenically unsaturated monomer.

Ethylenically unsaturated monomers containing at least one acid group or monomer bearing an acid group that may be used include α,β-ethylenic unsaturated carboxylic acids such as acrylic acid, methacrylic acid, crotonic acid, maleic acid and itaconic acid. (Meth)acrylic acid and crotonic acid are used in particular, and more particularly (meth)acrylic acid is used.

The esters of acid monomers are advantageously chosen from esters of (meth)acrylic acid (also known as (meth)acrylates), especially alkyl (meth)acrylates, in particular of a C₁-C₂₀ and more particularly a C₁-C₈ alkyl, aryl (meth)acrylates, in particular of a C₆-C₁₀ aryl, and hydroxyalkyl (meth)acrylates, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters are in particular alkyl (meth)acrylates.

According to the present invention, the alkyl group of the esters may be either fluorinated or perfluorinated, i.e. some or all of the hydrogen atoms of the alkyl group are replaced with fluorine atoms.

Examples of amides of the acid monomers that may be mentioned include (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide and N-t-octylacrylamide.

Examples of vinyl esters that may be mentioned include vinyl acetate, vinyl neodecanoate, vinyl pivalate, vinyl benzoate and vinyl t-butylbenzoate.

Styrene monomers that may be mentioned include styrene and α-methylstyrene.

The given list of monomers is not limiting, and it is possible to use any monomer known to those skilled in the art falling within the categories of acrylic and vinyl monomers (including monomers modified with a silicone chain).

Photoinitiator

The photoinitiators that may be used in the cosmetic compositions of the present invention are also known in the art and are described, for example, in the following articles, the content of which forms an integral part of the present patent application: “Les photoinitiateurs dans la réticulation des revêtements [Photoinitiators in the crosslinking of layerings]”, G. Li Bassi, Double Liaison—Chimie des Peintures, No. 361, November 1985, pages 34-41; “Applications industrielles de la polymérisation photoinduite [Industrial applications of photo-induced polymerization]”, Henri Strub, L'Actualité Chimique, February 2000, pages 5-13; and “Photopolymères: considérations théoriques et réaction de prise [Photopolymers: theoretical considerations and setting reaction]”, Marc, J. M. Abadie, Double Liaison—Chimie des Peintures, No. 435-436, 1992, pages 28-34.

These Photoinitiators Encompass:

the α-hydroxy ketones sold, for example, under the names Irgacure® 184, 1173, 2959, 149, 1000, 500 and 4265 by the company Ciba,

the α-amino ketones sold, for example, under the names Irgacure® 907 and 369 by the company Ciba,

the chloroacetophenones sold, for example, under the names Trigonal® P by the company Akzo and Sandoray® 1000 by the company Sandoz,

the aromatic ketones sold, for example, under the names Daitocure® by Dainippon, Uvecryl® P 36 by UCB, Esacure® TZT by Lamberti and Quantacure® ITX by Ward Blenkinsop. Mention may also be made of thioxanthones (for example Ultracure® DXT from Sherwin Williams) and quinones (2-ethylanthraquinone from BASF). These aromatic ketones usually require the presence of a hydrogen-donating compound such as tertiary amines and more particularly alkanolamines,

the benzoin ethers sold, for example, under the name Esacure® EB-3 by the company Lamberti and under the name Trigonal® 14 by Akzo,

the α-dicarbonyl derivatives, the most common representative of which is benzyl dimethyl ketal, sold under the name Irgacure® 651 by Ciba. Other commercial products are sold by the company Lamberti under the name Esacure® KBO and by the company Ward Blenkinsop under the name Quantacure® PDO,

the acylphosphine oxides such as, for example, the bis-acylphosphine oxides (BAPOs) sold, for example, under the names Irgacure® 819, 1700, 1800 and 1850 and Darocur® 4265 by the company Ciba.

One particular group of photoinitiators that is advantageous according to the invention is that of copolymerizable photoinitiators. These are molecules comprising both a photo-initiating group capable of a photo-induced free-radical splitting and at least one ethylenic double bond. The photoinitiators of this group have the advantage over the standard photoinitiators mentioned above of being able to be integrated, via the double bond, into the macromolecular system. This possibility reduces the concentration of free residual photoinitiators that have not undergone a photo-induced free-radical cleavage and consequently improves the harmlessness of the article according to the invention.

Examples of such copolymerizable photoinitiators that may be mentioned include the benzophenone acrylate derivatives sold by the company UCB under the names Ebecryl® P36 and Ebecryl® P37.

A mixture of photoinitiators that absorb light at different wavelengths is preferably used in the reactive system of the present invention. It is thus possible to adapt the absorption spectrum of the crosslinkable compositions to the emission spectrum of the light sources used.

The concentration of the photoinitiator(s) used depends on a large number of factors, for instance the reactivity of the various components of the mixture, the presence of pigments or dyes, the desired crosslinking density, the intensity of the light source or the exposure time.

To obtain satisfactory staying power properties, a total amount of photoinitiator(s) at least equal to 0.1% by weight and of not more than 10% by weight, and preferably between 0.2% and 5% by weight, relative to the total weight of compounds (A) and (B) comprising ethylenic double bonds, will generally be used.

In another variant of the invention, the crosslinking may be performed by placing in contact compounds (A) and/or (B) bearing functions (X) and/or (Y) in a blocked form that can be unblocked beforehand or under the reaction conditions adopted for the crosslinking.

This unblocking may be performed, for example, under the action of water in the presence or absence of catalyst, especially when Y is chosen from blocked amine functions and when X is a reactive function such as an isocyanate or an epoxide function.

It may also be performed under the action of heat in the presence or absence of catalyst, especially when X is chosen from blocked isocyanate functions and when Y is a function comprising labile hydrogens, for instance an amine or hydroxyl function.

Similarly, this unblocking may be performed under the action of radiation (for example UV, RX, laser, etc.) optionally in the presence of a photoinitiator, when X and Y are chosen from the reactive functions such as unsaturated ethylenic double bonds.

Blocked Isocyanate Functions

The blocked isocyanate functions capable of reacting, after heat activation, with the functions containing labile hydrogen, preferably correspond to the following formula:
—NH—C(═O)—M
in which M represents a radical derived from a blocking agent MH chosen from organic compounds comprising one or more, and particularly only one labile hydrogen atom.

The blocking agents must be capable of preventing the subsequent reaction of the isocyanate groups at room temperature, or more generally at a temperature below 45° C., with any other molecule containing labile hydrogen atoms, but must allow this reaction at a higher temperature, that is to say generally greater than or equal to 50° C., after thermal unblocking of the isocyanate function.

Among the sold compounds containing blocked isocyanate groups that may be mentioned are those sold under the names Vestanat® B1358A, Vestanat® B1370, Vestanat® B1358/100 by the company Creanova, under the names Tolonate® D2 or D2R565 by the company Rhodia, under the name Desmodur® Z4470 by the company Bayer and under the names Trixene® B1 7951 and Trixene® B1 7982 by the company Baxenden.

It is also possible to use, as compounds (A) or (B), compounds containing “self-blocked” isocyanate functions, such as urethane-diones, obtained by dimerizing 2 molecules of diisocyanates, or alternatively tris((C₁₋₆ alkoxy)carbonylamino)triazines such as the product of condensation of melanine, of dimethyl carbonate and of butanol. Such compounds are especially sold under the name Cylink® 2000 by the company Cyteck.

Functions Containing Blocked Labile Hydrogens

The functions containing blocked labile hydrogens are especially chosen from amine functions blocked in ketimine and aldimine form, and amino alcohol functions blocked in oxazolidine form.

The compounds bearing blocked amine functions may be chosen especially from:

a) the blocked polyamines of general formula: 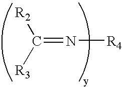
in which:

y ranges from 2 to 100,

R₂ is equal to or different than R₃ and chosen from a hydrogen atom and alkyl groups containing from 1 to 4 carbon atoms.

Compounds containing amine functions blocked in ketimine form are especially sold under the name Epikure® H3 and Epikure® 3505 by the company Shell, and Vestamin® A139 by the company Creanova.

According to one particular embodiment of the invention, the reactive system may comprise at least one compound comprising both unblocked reactive functions X and blocked reactive functions Y.

Compounds containing amino alcohol functions blocked in oxazolidine form are especially described in documents WO-A-99/07763, JP-A-09-241501, WO-A-96/20231, WO-A-95/14528, U.S. Pat. No. 5,126,421, U.S. Pat. No. 4,381,388 and U.S. Pat. No. 4,504,647. They are sold under the names Incozol® 4 and Incozol® LV by the company Industrial Copolymer Ltd., Hardener® OZ by the company Bayer and Zoldine® RD-4 by the company Angus Chemicals Co.

The functions containing blocked labile hydrogens may also be featured by:

cyclic groups capable of reacting by ring opening, for instance:
anhydride functions capable of generating a carboxylic function to react in turn with a reactive function or a function containing labile hydrogen: the anhydrides preferentially used are chosen from aliphatic, cycloaliphatic and aromatic anhydrides. Examples of anhydrides include succinic anhydride, methylsuccinic anhydride, dodecenylmethylsuccinic anhydride, phthalic anhydride, maleic anhydride and itaconic anhydride,
lactone and lactam functions, for instance ε-caprolactone and ε-caprolactam, and

acetoacetate functions: R—O—C═O—CH_{2-c}═O—CH₃ which are capable of reacting in the presence of formaldehyde (or of melamine/formaldehyde, urea/formaldehyde or alkoxylated resins), isocyanate functions, epoxide functions, unsaturated ethylenic double bonds (Michael reaction) or amine functions.

The reactive systems of the present invention may contain one or more catalysts capable of accelerating the crosslinking reaction.

These catalysts are especially chosen from tertiary amines such as diazabicyclo[2.2.2]octane, quinuclidine and 3,3,6,9,9-pentamethyl-2,10-diazabicyclo[4.4.0]dec-1-ene, tin chloride, organometallic compounds such as metal acetonylacetates, organometallic tin compounds, calcium hexanoate, calcium 2-ethylhexanoate, calcium octanoate and calcium linoleate, dibutyltin dilaurate, bismuth tris(2-ethylhexanoate) and zinc bis(2-ethylhexanoate).

According to the present invention, the concentration of catalysts is preferably between 0.1% and 5% by weight and more particularly between 0.2% and 3% by weight relative to the total weight of the compound bearing blocked isocyanate groups.

The crosslinkable composition according to the present invention may contain one or more solvents chosen from water and organic solvents, among which mention may be made of:

b) ketones that are liquid at room temperature, such as methyl ethyl ketone, methyl isobutyl ketone, diisobutyl ketone, isophorone, cyclohexanone and acetone,

c) alcohols that are liquid at room temperature, such as ethanol, isopropanol, diacetone alcohol, 2-butoxyethanol and cyclohexanol,

d) glycols that are liquid at room temperature, such as ethylene glycol, propylene glycol, pentylene glycol and glycerol,

e) propylene glycol ethers that are liquid at room temperature, such as propylene glycol monomethyl ether, propylene glycol monomethyl ether acetate and dipropylene glycol mono-n-butyl ether,

f) short-chain esters (containing in total from 3 to 8 carbon atoms), such as ethyl acetate, methyl acetate, propyl acetate, n-butyl acetate and isopentyl acetate,

g) alkanes that are liquid at room temperature, such as decane, heptane, dodecane and cyclohexane,

h) aromatic hydrocarbons that are liquid at room temperature, such as toluene and xylene,

i) silicones that are liquid at room temperature, and

j) mixtures thereof.

The solvent content of the composition may range from 0.1% to 80% by weight and preferably from 1% to 60% by weight relative to the total weight of the initial composition before crosslinking.

According to one particular mode, the crosslinkable composition may be free of solvent.

The crosslinkable compositions used in the invention may also comprise various additives, with the proviso that these additives are not capable of interfering with the crosslinking reaction.

In particular, the crosslinking may be performed in the presence of at least one film-forming agent and especially nitrocellulose and/or cellulose esters.

Solid Particles

The articles according to the invention may also contain, especially in the crosslinked film(s), solid particles especially in an amount that is effective for improving the staying power and in particular the wear resistance of the article. These particles may be formed from the materials chosen from polymeric or nonpolymeric mineral materials, polymeric or nonpolymeric organic materials, which may be metallic, composite materials, and mixtures thereof.

In particular, the particles that may be used in the articles according to the invention may comprise mineral elements or compounds known in the field. Suitable particles may be formed from ceramic materials, metallic materials or mixtures thereof. Suitable ceramic materials comprise metal oxides, metal nitrides, metal carbides, metal sulfides, metal silicates, metal nitrides and metal carbonates, and mixtures thereof.

Nonpolymeric mineral materials may be chosen from graphite, metals, oxides, carbides, nitrides, porides, sulfides, silicates, carbonates, sulfates and hydroxides. By way of illustration of a mineral oxide, an example that may be mentioned is zinc oxide. By way of illustration of inorganic sulfides, examples that may be mentioned include molybdenum disulfide, tantalum disulfide, tungsten disulfide and zinc disulfide. Nonlimiting examples of mineral silicates that may be mentioned include aluminum magnesium silicates such as vermiculite. Examples of suitable metals that may be mentioned include molybdenum, platinum, palladium, nickel, aluminum, gold, iron and silver, and alloys and mixtures thereof.

According to one particular embodiment, the particles may be particles chosen from fumed silica, especially the family known as Aerosil® from Degussa, amorphous silica, colloidal silica, alumina, colloidal alumina, titanium dioxide, yttrium oxide, colloidal zirconium and clays, and mixtures thereof.

Intercalated or even exfoliated phyllosilicates are most particularly suitable as clays. The term “intercalated” means that these phyllosilicates have been treated with organic or inorganic compounds in order to introduce molecules of these compounds into the interfoliar spaces. Exfoliated phyllosilicates denote separated platelets generally obtained by shearing intercalated phyllosilicates. Such materials are especially described in documents WO 93/04118, U.S. Pat. No. 5,721,306 and U.S. Pat. No. 6,500,411.

The particles may also be formed from a mineral polymeric material. Mineral polymeric materials that may especially be mentioned include polyphosphazenes, polysilanes, polysiloxanes, polymeric sulfides, polymeric seleniums and silicones, and mixtures thereof. A particular example of particles formed from a mineral polymeric material consists of the particles sold under the name Tospearl by the company Toshiba Silicone Company, which are crosslinked siloxane particles.

The particles may also be formed from nonpolymeric organic materials. Nonpolymeric organic materials that may especially be mentioned include stearates, such as zinc stearate and aluminum stearate, diamond, carbon black and stearamide.

The particles may also be hollow particles formed from polymeric materials chosen from polymeric or nonpolymeric mineral materials, polymeric or nonpolymeric organic materials and composite materials, and mixtures thereof. An example of suitable materials forming hollow particles that may especially be mentioned consists of hollow glass particles.

Other examples of particles including colloidal silicas that may especially be mentioned are the particles sold by the company Nissan Chemical Company under the name “Organosilicasol®” such as Organosilicasol MT-ST, and by the company Clariant Corporation under the name “High link®”, colloidal. aluminas such as those sold by the company Nalco Chemical under the name “Nalco 8667®” and colloidal zirconias such as those sold by the company Nissan Chemical Company under the name “HIT-32M®”.

More particularly, these particles may be chosen from talc, zinc stearate, mica, kaolin, polyamide (Nylon®) powders (Orgasole from Atochem), polyethylene powders, tetrafluoroethylene polymer (Teflon®) powders, starch, boron nitride, polymer microspheres such as those of polyvinylidene chloride/acrylonitrile, for instance Expancel® (Nobel Industrie) or of acrylic acid copolymers (Polytrap® from the company Dow Corning), silicone resin microbeads (for example Tospearls® from Toshiba) and elastomeric organo-polysiloxanes, and mixtures thereof.

In particular, the content of particles in the articles according to the invention is less than 40% by weight, especially less than 25%, more particularly less than 15% and even less than 10% by weight relative to the total weight of the article.

Adhesive Material

The article according to the invention has an adhesive outer face. Such an adhesive face is generally obtained by virtue of the presence of at least one coat of at least one adhesive material.

For the purposes of the present invention, the term “material” means a polymer or a polymeric system that may comprise one or more polymers of different nature. This adhesive material may be in the form of a polymer solution or a dispersion of polymer particles in a solvent. This adhesive material may also contain a plasticizer. This adhesive material should have a certain tack power defined by its viscoelastic properties.

The viscoelastic properties of a material are conventionally defined by two characteristic values, which are the following:

the elastic modulus, which represents the elastic behavior of the material for a given frequency and which is conventionally noted as G′,

the viscous modulus, which represents the viscous behavior of the material for a given frequency and which is conventionally noted as G″.

These magnitudes are especially defined in the “Handbook of Pressure Sensitive Adhesive Technology” 3rd edition, D. Satas, chapter 9, pp. 155 to 157.

The adhesive materials that may be used according to the present invention have viscoelastic properties that are measured at a reference temperature of 35° C. and within a certain frequency range.

In the case of adhesive materials in the form of a solution or dispersion of polymer in a volatile solvent (such as water, a short ester, a short alcohol, acetone, etc.), the viscoelastic properties of this material are measured under conditions in which it has a volatile solvent content of less than 30% and in particular a volatile solvent content of less than 20%.

The elastic modulus of the material is measured in particular at three different frequencies:

at low frequency, i.e. at 2×10⁻² Hz,

at an intermediate frequency, i.e. at 0.2 Hz,

at high frequency, i.e. at 2 Hz, and the viscous modulus is measured at a frequency of 0.2 Hz.

These measurements make it possible to evaluate the change in the tack powder of the adhesive material over time.

These viscoelastic properties are measured during dynamic tests under low-amplitude sinusoidal stresses (small deformations) performed at 35° C. over a frequency range of from 2×10⁻² to 20 Hz on a “Haake RS50®” rheometer under a torsion/shear stress, for example in cone-plate geometry (for example with a cone angle of 1°).

Advantageously, said adhesive material satisfies the following conditions:

G′(2 Hz, 35° C.)≧10³ Pa, and

G′(35° C.)≦10⁸ Pa, in particular G′(35° C.)≦10⁷ Pa,

G′(2×10⁻² Hz, 35° C.)≦3×10⁵ Pa, in which:

G′(2 Hz, 35° C.) is the elastic shear modulus of said adhesive material, measured at a frequency of 2 Hz and at a temperature of 35° C.,

G′(35° C.) is the elastic shear modulus of said adhesive material measured at a temperature of 35° C., for any frequency between 2×10⁻² and 2 Hz,

G′(2×10⁻² Hz, 35° C.) is the elastic shear modulus of said adhesive material, measured at a frequency of 2×10⁻² Hz and at a temperature of 35° C.

In one particular form of the invention, the adhesive material also satisfies the following condition:

G″/G′(0.2 Hz, 35° C.)≧0.35

in which:

G″(0.2 Hz, 35° C.) is the viscous shear modulus of said adhesive material, measured at a frequency of 0.2 Hz and at a temperature of 35° C.,

G′(0.2 Hz, 35° C.) is the elastic shear modulus of said adhesive material, measured at a frequency of 0.2 Hz and at a temperature of 35° C.

In one particular form of the invention:

G′(2 Hz, 35° C.)≧5×10³ Pa, and in particular G′(2 Hz, 35° C.) ≧10⁴ Pa.

In another particular form of the invention:

G′(2×10⁻² Hz, 35° C.)≦5×10⁴ Pa.

In particular, the adhesive materials according to the invention satisfy the following four conditions:

G′(2 Hz, 35° C.)≧10⁴ Pa, and

G′(35° C.)≦10⁸ Pa, in particular G′(35° C.) ≦10⁷ Pa,

G′(2×10⁻² Hz, 35° C.)≦5×10⁴ Pa, and

G″/G′(0.2 Hz, 35° C.)≧0.35.

In general, the adhesive is such that said article cannot be removed by peeling when it is applied to the surface of a synthetic or natural nail after an adhesion time of at least 24 hours.

More particularly, the adhesive materials according to the invention may be chosen from adhesives of “Pressure Sensitive Adhesives” type, for instance those cited in the “Handbook of Pressure Sensitive Adhesive Technology” 3rd edition, D. Satas.

The adhesive materials according to the invention are especially polymers chosen from block or statistical copolymers comprising at least one monomer or a combination of monomers whose resulting polymer has a glass transition temperature of less than room temperature (25° C.), these monomers or associations of monomers possibly being chosen from butadiene, ethylene, propylene, isoprene, isobutylene and a silicone, and mixtures thereof. Examples of such materials are block polymers of styrene-butadiene-styrene, styrene-(ethylene-butylene)-styrene or styrene-isoprene-styrene type, for instance those sold under the trade names “Kraton®” from SHELL CHEMICAL Co. or “Vector®” from EXXON.

The adhesive materials according to the invention are particular adhesive polymers chosen from:

polyurethanes,

acrylic polymers,

silicones,

butyl rubbers, especially chosen from polyisobutylenes,

ethylene-vinyl acetate polymers,

polyamides optionally modified with fatty chains,

natural rubbers,

and mixtures thereof.

They may in particular be adhesive copolymers derived from the copolymerization of vinyl monomers with polymeric species, for instance those described in patent U.S. Pat. No. 6,136,296. Adhesive copolymers that may also be suitable for the invention are those described in patent U.S. Pat. No. 5,929,173, having a polymer backbone, with a Tg ranging from 0° C. to 45° C., grafted with chains derived from acrylic and/or methacrylic monomers and having, in contrast, a Tg ranging from 50° C. to 200° C.

The adhesive materials are chosen, for example, from polyisobutylenes with a relative molar mass Mv greater than or equal to 10000 and less than or equal to 150000. In particular, this relative molar mass is greater than or equal to 18000 and less than or equal to 150000.

As commercial products that are particularly suitable for the present invention, mention may be made of polyisobutylenes with respective relative molar masses Mv of 40000, 55000 and 85000 sold under the respective trade names “Oppanol B 10®”, “Oppanol B 12®” and “Oppanol B 15®” by the company BASF, and mixtures thereof.

The adhesive material in the article in accordance with the invention is generally in the form of a layer with a thickness of from 1 micron to 100 microns, in particular from 1 micron to 50 microns and preferably from 1 micron to 25 microns.

According to one particular embodiment of the invention, the layer formed by the adhesive material is directly in contact with the crosslinked film.

Advantageously, the adhesive material and the film are compatible by virtue of their chemical nature. Specifically, as stated previously, the solvent for the adhesive is capable of leading to an increase in the mass of the crosslinked film placed in contact therewith, especially by at least 10% by weight relative to the initial weight of the crosslinked film. This increase is reflected more specifically by a gain in mass of the film.

However, as stated previously, the article may also have a film of varnish between the adhesive layer and the crosslinked film. This intermediate layer between the layer of adhesive material and the film may be obtained by evaporating the aqueous phase of the aqueous or organic dispersion of a dispersion or solution of at least one film-forming polymer. Such a layer may consist in particular of a film of varnish, which is especially colored. Such architecture is particularly advantageous in terms of duration over time. The crosslinked film effectively protects the film of varnish against impacts and thus significantly prolongs its staying power over time.

Other Additives

The article may also comprise, especially in its crosslinked film, at least one dyestuff, especially such as a pigment and/or at least one nacre and/or at least flakes conventionally used in cosmetic compositions. Needless to say, some of these compounds may also be illustrated by the solid particles discussed previously.

The term “pigments” should be understood as meaning white or colored, mineral or organic particles, which are insoluble in an aqueous solution, and which are intended to color and/or opacify the resulting film. The term “nacres” should be understood as meaning iridescent particles, especially produced by certain molluscs in their shell or else synthesized. These nacres serve especially to modify the texture of the expected film.

The pigments may be present in a proportion of from 0.01% to 15% by weight, especially from 0.01% to 10% by weight and in particular from 0.02% to 5% by weight relative to the total weight of the article. As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

Among the organic pigments that may be used in the invention, mention may be made of carbon black, pigments of D & C type, and lakes based on cochineal carmine or on barium, strontium, calcium or aluminum, or alternatively the diketopyrrolopyrroles (DPP) described in documents EP-A-542669, EP-A-787730, EP-A-787731 et WO-A-96/08537.

The nacres may be present in a proportion of from 0.01% to 15% by weight, preferably from 0.01% to 10% by weight and better still from 0.02% to 5% by weight relative to the total weight of the article. The nacreous pigments may be chosen from white nacreous pigments such as mica layered with titanium or with bismuth oxychloride, colored nacreous pigments such as titanium mica with iron oxides, titanium mica especially with ferric blue or chromium oxide, titanium mica with an organic pigment of the abovementioned type and also nacreous pigments based on bismuth oxychloride.

These nacres, and also flakes or other compounds with reflective effects, are particularly advantageous for enhancing the natural gloss of the crosslinked film.

The article according to the invention may also comprise water-soluble or liposoluble dyes in a content ranging from 0.01% to 10% by weight and especially ranging from 0.01% to 5% by weight relative to the total weight of the film. The liposoluble dyes are, for example, Sudan red, DC Red 17, DC Green 6, β-carotene, soybean oil, Sudan brown, DC Yellow 11, DC Violet 2, DC Orange 5 or quinoline yellow. The water-soluble dyes are, for example, beetroot juice or methylene blue.

It may also contain ingredients commonly used in cosmetics and more especially in nail cosmetics and/or nail care. They may be chosen especially from vitamins, trace elements, softeners, sequestrants, acidifying or basifying agents, film-forming polymers, spreading agents, wetting agents, thickeners, dispersants, antifoams, preserving agents, UV-screening agents, active agents, moisturizers, fragrances, neutralizers, stabilizers and antioxidants, and mixtures thereof.

Thus, when the articles according to the invention are more particularly intended for caring for natural nails, they may especially incorporate, as active agents, hardeners for keratin materials, active agents acting on the growth of the nails, for instance methylsulfonylmethane, and/or active agents for treating various complaints located on the nails, for instance onichomycosis.

The amounts of these various ingredients are those conventionally used in this field, for example from 0.01% to 20% and especially from 0.01% to 10% by weight relative to the total weight of the article.

The article in accordance with the invention generally has a thickness of from 1 micron to 500 microns, especially from 1 micron to 300 microns and in particular from 1 micron to 200 microns.

As mentioned previously, the article in accordance with the invention is layered at least on its adhesive outer face with a removable support.

Such a support may be of any nature which is compatible with the fact that, although it is in contact with an adhesive material, it can nevertheless be separated therefrom.

The removable support defined previously may especially be in the form of a protective layer consisting, for example, of a film, in particular of a plastic film or a paper or a textile structure of sheet type.

Advantageously, this support consists of a transparent material in order to prevent any error in the choice of the color. It may consist of one or more layers that may be of different nature. For example, it may be a sheet of paper layered with one of the plastics mentioned hereinbelow.

As suitable plastic film that may be used, for example, in the article in accordance with the invention, mention may be made of films made of polyesters, for example polyethylene terephthalates, polybutylene terephthalates or polyethylene sebacates, or made of polyethylene, polypropylene or polyamides such as polyhexamethylene adipate, polycaprolactam or poly(ω-undecanoic acid amide). On account of their surface characteristics, these plastics are, of course, not removable per se. In order to give them this property, it is necessary to perform a surface treatment using suitable substances, such as a treatment with silicones, or, particularly advantageously, a treatment with salts of long-chain fatty acids, for instance of C₁₂ to C₂₂, these acids being saturated or possibly containing up to three olefin bonds, and at least divalent metals, in particular salts of heavy transition metals of this type and in particular chromium salts.

The textile structure of sheet type may be a woven or a nonwoven.

According to one particular embodiment, the article in accordance with the invention is layered on both faces with an identical or different removable support.

The article in accordance with the present invention may be in various forms, such as a star, a square, a circle, etc.

As indicated previously, the present invention also relates to a process for preparing a flexible article for making up and/or caring for the nails. Such an article may especially be obtained with the device described in patent U.S. Pat. No. 4,903,840.

More specifically, this process comprises a step of crosslinking of at least two compounds (A) and (B) as defined above so as to obtain a film. This crosslinking may be performed according to conventional methods that are well known to those skilled in the art.

The adhesive material is generally deposited in the form of a layer of material in a thickness that may range from 0.5 micron to 200 microns and in particular from 1 micron to 100 microns.

The article obtained, and in particular the excess of film, is then generally cut, before or after its application, to the desired size and shape with small scissors, a nail clipper or by scratching the film.

The present invention also relates to a process for making up the nails in which the article as defined previously is applied.

The makeup thus obtained may be removed using makeup removers that are common in the field of nail varnishes.

1-47. (canceled)

48. A flexible article for making up and/or caring for the nails and/or false nails, comprising:
at least one adhesive layer for fixing the article to the nail, and
at least one crosslinked film.

49. An article according to claim 48, wherein it further comprises, between the adhesive layer and the crosslinked film, at least one film of colored varnish.

50. An article according to claim 48, wherein said crosslinked film is transparent.

51. An article according to claim 49, wherein the film of varnish is obtained by crosslinking or evaporating the organic or aqueous solvent phase from a solution or dispersion of at least one film-forming polymer.

52. An article according to claim 51, wherein the film-forming polymer is chosen from nitrocellulose and cellulose esters.

53. An article according to claim 48, wherein it has a solids content of greater than 80% by weight relative to its total weight.

54. An article according to claim 48, wherein it has an uptake of water brought to 25° C. of less than or equal to 20%.

55. An article according to claim 48, wherein it has a storage modulus E′ of greater than or equal to 1 MPa, at a temperature of 30° C. and a frequency of 0.1 Hz.

56. An article according to claim 48, wherein it has an ultimate strain εᵣ of greater than or equal to 5%, and/or an energy at break per unit volume Wᵣ of greater than or equal to 0.2 J/cm³.

57. An article according to claim 48 wherein the crosslinking of the crossinked film is performed thermally, photochemically and/or chemically, in the presence or absence of a catalyst.

58. An article according to claim 48, wherein said crosslinking of the crossinked film is of polyaddition and/or polycondensation type.

59. An article according to claim 57, wherein the crosslinked film is derived from the crosslinking of a reactive system formed by:
at least one first compound (A) comprising at least two functions X,
at least one second compound (B) comprising at least two functions Y that are reactive with the functions X.

60. An article according to claim 59, wherein said reactive system has a mean functionality (total number of functions X and Y/total number of molecules of compounds (A) and (B)) of greater than 2.

61. An article according to claim 59, wherein said compound (A) and/or compound (B) is organic and is chosen among oligomers, polymers or copolymers.

62. An article according to claim 59, wherein said compound (A) and/or compound (B) is inorganic and bears at the surface said functions X or Y.

63. An article according to claim 59, wherein said functions X and Y are chosen from “reactive” functions and functions comprising at least one labile hydrogen.

64. An article according to claim 59, wherein the reactive functions are chosen from isocyanate, epoxide and ethylenic double bond functions.

65. An article according to claim 63, wherein the functions containing labile hydrogen(s) are of the carboxylic, alcohol, primary or secondary amine, amide, amino alcohol and/or thiol type.

66. An article according to claim 58, wherein said crosslinking uses at least one first type of compound comprising reactive functions and a second type of compound comprising functions containing labile hydrogen(s).

67. An article according to claim 66, wherein the reactive functions are epoxide and/or isocyanate functions.

68. An article according to claim 64, wherein the compounds bearing reactive functions of isocyanate type are chosen from aliphatic, cycloaliphatic and aromatic diisocyanates, triisocyanates and polyisocyanates with a molecular mass of less than 10000.

69. An article according to claim 68, wherein said compounds are chosen from:
1,4-tetramethylene diisocyanate, 1,6-hexamethylene diisocyanate, 2,6- and 2,4-toluene diisocyanate, diphenylmethane diisocyanate and isophorone diisocyanate,
the triisocyanates of formulae: 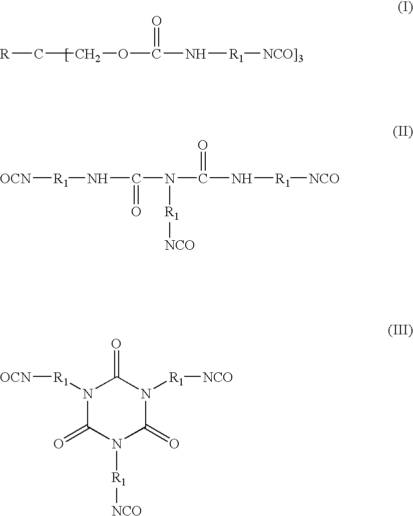
with R being an alkyl radical containing from 1 to 30 carbon atoms, and each R₁ independently representing a linear, branched or cyclic divalent hydrocarbon-based radical containing from 2 to 30 carbon atoms,
polycondensates containing isocyanate end or side groups, such as polyurethanes, polyureas, polyesters, polyamides, polyepoxy, polyethers and/or perfluoropolyethers, and
polymers resulting from the copolymerization of vinyl, allylic and/or (meth)acrylic monomers and of ethylenically unsaturated comonomers comprising a free isocyanate function.

70. An article according to claim 64, wherein the compounds bearing reactive functions of epoxide type are chosen from:
bisphenol A diglycidyl ether,
diepoxy resins,
epoxy ester resins containing α,ω-diepoxy end groups,
epoxy ether resins containing α,ω-diepoxy end groups,
natural or synthetic oils bearing at least two epoxide groups,
oligomers or polymers resulting from the copolymerization of unsaturated or vinyl, allylic and/or (meth)acrylic monomers, and of ethylenically unsaturated comonomers comprising a free epoxide function, and
polycondensates containing epoxy end and/or side groups.

71. An article according to claim 65, wherein the compounds bearing functions containing labile hydrogen(s) are chosen from:
aliphatic diols,
polyols,
aliphatic, cycloaliphatic or aromatic diamines,
multifunctional amines containing more than two amine groups,
oligomers bearing at least two amine groups,
alkyd resins, and
dendrimers or hyperbranched polymers whose chain end groups are primary amines.

72. An article according to claim 48, wherein said crosslinking of the crossinked film is performed photochemically and involves at least two compounds (A) and (B) bearing functions of unsaturated double bond type in the presence of a photoinitiator.

73. An article according to claim 72, wherein (A) and (B) are chosen so as to form a reactive system whose mean valency is greater than 2.

74. An article according to claim 73, wherein compounds (A) and (B) are chosen from:
a) ethylenically unsaturated polyesters,
b) polyesters containing (meth)acrylate side and/or end groups,
c) polyurethanes and/or polyureas containing (meth)acrylate groups,
d) poly(C₁₋₅₀ alkyl) (meth)acrylates comprising at least two ethylenic double bond functions borne by the hydrocarbon-based side and/or end chains,
e) polyorganosiloxanes containing (meth)acrylate or (meth)acrylamide groups,
f) dendrimers and hyperbranched polymers bearing (meth)acrylate or (meth)acrylamide end groups, and
g) ethylenically unsaturated monomers.

75. An article according to claim 48, wherein said film also comprises a catalyst.

76. An article according to claim 48, wherein it also comprises organic or inorganic solid particles.

77. An article according to claim 48, wherein said adhesive layer comprises at least one adhesive material.

78. An article according to claim 48, wherein said adhesive material is such that said article cannot be removed by peeling when it is applied to the surface of a synthetic or natural nail after an application time of at least 24 hours.

79. An article according to claim 77, wherein said adhesive material is chosen from copolymers derived from the copolymerization of vinyl monomers with polymeric species, copolymers bearing a polymer backbone, with a Tg ranging from 0° C. to 45° C., grafted with chains derived from acrylic and/or methacrylic monomers and having, in contrast, a Tg ranging from 50° C. to 200° C. and polyisobutylenes with a relative molar mass Mv of greater than or equal to 10000 and less than or equal to 150000.

80. An article according to claim 48, wherein said adhesive layer is layered at the surface with a removable support constituted by a plastic film modified by means of a surface treatment with silicone or with salts of C₁₂ to C₂₂ fatty acids.

81. A product for making up and/or caring for the nails and/or false nails, comprising, in a substantially airtight packaging, at least one article according to claim 48, the packaging being such that said article is preserved in a partially dry form.

82. A product according to claim 81, wherein said article has a solids content of less than 80%, by weight relative to the total weight of said article.

83. A product according to claim 81, wherein the packaging comprises a reservoir capable of containing said article in a leaktight manner.

84. A method for preparing a flexible article for making up and/or caring for the nails, comprising at least the steps consisting in superposing on a removable support:
a) at least one layer of a composition based on at least one adhesive material, and
b) at least one layer of a crosslinkable composition, the crosslinking of said composition being performed consecutively to its deposition so as to obtain a crosslinked film.

85. A method according to claim 84, wherein a film of colored varnish is also formed between the adhesive layer and the crosslinked film.

86. A method according to claim 85, wherein the film of varnish is obtained by crosslinking and/or evaporating the organic or aqueous solvent phase from a solution or dispersion of at least one film-forming polymer.

87. A method for preparing a flexible article for making up and/or caring for the nails, comprising at least the steps consisting in superposing on a removable support:
a) at least one layer of a composition based on at least one adhesive material, and
b) at least one layer of a crosslinkable composition, the crosslinking of said composition being performed consecutively to its deposition so as to obtain a crosslinked film,
wherein the crosslinking is of polyaddition and/or polycondensation type as defined in claim 59.

88. A method for preparing a flexible article for making up and/or caring for the nails, comprising at least the steps consisting in superposing on a removable support:
a) at least one layer of a composition based on at least one adhesive material, and
b) at least one layer of a crosslinkable composition, the crosslinking of said composition being performed consecutively to its deposition so as to obtain a crosslinked film,
wherein the crosslinking is performed photochemically as defined in claim 72.

89. A method for preparing a flexible article for making up and/or caring for the nails, comprising at least the steps consisting in superposing on a removable support:
a) at least one layer of a composition based on at least one adhesive material, and
b) at least one layer of a crosslinkable composition, the crosslinking of said composition being performed consecutively to its deposition so as to obtain a crosslinked film,
wherein the adhesive material is as defined in claim 78.

90. A method for preparing a product according to claim 81, comprising the steps consisting in superposing on a removable support:
a) at least one layer of a composition based on at least one adhesive material,
b) at least one layer of a crosslinkable composition, the crosslinking of said film being performed consecutively to the deposition of said composition,
c) if necessary, partial drying of the article thus obtained, and
d) packaging of said article in a partially dry form in substantially airtight packaging.

91. A cosmetic method for making up the nails using an article, comprising the fact that the adhesive face of an article as defined in claim 48 is applied to a natural or synthetic nail.
